(19) United States
(12) Patent Application Publication
(10) Pub. No.: US 2015/0246978 A1
(43) Pub. Date: Sep. 3, 2015

(54) PROCESSES AND APPARATUS FOR MANAGING AND RECYCLING SULFUR DIOXIDE IN BIOREFINERIES

(71) Applicant: API Intellectual Property Holdings, LLC, Atlanta, GA (US)

(72) Inventor: Mark SZCZEPANIK, Alpena, MI (US)

(21) Appl. No.: 14/621,370

(22) Filed: Feb. 12, 2015

Related U.S. Application Data
(60) Provisional application No. 61/945,848, filed on Feb. 28, 2014.

(51) Int. Cl. C08B 37/00; C12P 19/02

(52) U.S. Cl. CPC C08B 37/0003; C12P 19/02

(57) ABSTRACT

What is disclosed is a biorefining process and system including the steps of extracting, hydrolyzing, and/or digesting a lignocellulosic biomass feedstock, or a component thereof, with a liquid or vapor solution comprising reactant sulfur dioxide or a derivative thereof, to generate cellulose-rich solids and an intermediate liquid stream; combusting or gasifying a sulfur-containing fuel to generate heat and an exhaust gas stream comprising produced sulfur dioxide; contacting the exhaust gas stream with the intermediate liquid stream to dissolve the produced sulfur dioxide into the intermediate liquid stream; and then recycling the intermediate liquid stream to reuse the produced sulfur dioxide as the reactant sulfur dioxide. The sulfur-containing fuel may be sulfonated lignin generated by reaction of lignin (derived from the feedstock) with the reactant sulfur dioxide, resulting in a closed loop for sulfur dioxide in the biorefinery.

### PRIORITY DATA

This patent application is a non-provisional application claiming priority to U.S. Provisional Patent App. No. 61/945,848, filed Feb. 28, 2014, which is hereby incorporated by reference herein.

### STATEMENT REGARDING FEDERALLY SPONSORED RESEARCH OR DEVELOPMENT

This invention was made with U.S. Government support under Contract No. DE-EE0002868. The U.S. Government has certain rights in this invention.

### FIELD

The present invention generally relates to biorefining processes utilizing sulfur dioxide for converting biomass into fermentable sugars, cellulose, and lignin.

### BACKGROUND

Biomass refining (or biorefining) is becoming more prevalent in industry. Cellulose fibers and sugars, hemicellulose sugars, lignin, syngas, and derivatives of these intermediates are being used by many companies for chemical and fuel production. Indeed, we now are observing the commercialization of integrated biorefineries that are capable of processing incoming biomass much the same as petroleum refineries now process crude oil. Underutilized lignocellulosic biomass feedstocks have the potential to be much cheaper than petroleum, on a carbon basis, as well as much better from an environmental life-cycle standpoint.

Lignocellulosic biomass is the most abundant renewable material on the planet and has long been recognized as a potential feedstock for producing chemicals, fuels, and materials. Lignocellulosic biomass normally comprises primarily cellulose, hemicellulose, and lignin. Cellulose and hemicellulose are natural polymers of sugars, and lignin is an aromatic/aliphatic hydrocarbon polymer reinforcing the entire biomass network. Some forms of biomass (e.g., recycled materials) do not contain hemicellulose.

It has been found that sulfur dioxide can be a very effective chemical for various biorefining processes, including fractionation of biomass with SO₂, water, and a solvent for lignin (AVAP® technology). Also, SO₂ may be utilized to hydrolyze extracted hemicellulose oligomers from biomass, to generate monomeric sugars, in a variation of the Green Power+® technology. The AVAP and Green Power+ technologies have been developed by American Process, Inc. and are commonly owned with the assignee of this patent application.

Improvements are still desired in overall management of SO₂ in biorefineries, as well as economic recovery and recycle of SO₂ or its derivatives. For example, there is a desire to reduce or eliminate the need for purchasing and storing pure sulfur dioxide. There is also a desire to improve the environmental air emissions of SO₂ from power boilers, when using any fuels containing sulfur.

### SUMMARY

The present invention addresses the aforementioned needs in the art.

In some variations, the invention provides a biorefining process comprising:

(a) providing a lignocellulosic biomass feedstock;

(b) extracting, hydrolyzing, and/or digesting the feedstock, or a component thereof, with a liquid or vapor solution comprising reactant sulfur dioxide or a derivative thereof, to generate cellulose-rich solids and an intermediate liquid stream;

(c) combusting or gasifying a sulfur-containing fuel to generate heat and an exhaust gas stream comprising produced sulfur dioxide;

(d) optionally removing at least a portion of hemicellulosic sugars and/or lignin, if present, from the intermediate liquid stream;

(e) contacting at least a portion of the exhaust gas stream with the intermediate liquid stream to dissolve the produced sulfur dioxide into the intermediate liquid stream; and

(f) recycling at least some of the intermediate liquid stream back to step (b) to reuse the produced sulfur dioxide as the reactant sulfur dioxide.

In some embodiments, the sulfur-containing fuel comprises coal. In these or other embodiments, the sulfur-containing fuel comprises an intermediate stream or product derived from a biomass source, such as biochar or lignin. The biomass source may be the same as or different than the lignocellulosic biomass feedstock.

In certain embodiments, the sulfur-containing fuel comprises sulfonated lignin. The sulfonated lignin may be provided from an external process, or it may be generated in step (b) by reaction of lignin (derived from the feedstock) with the reactant sulfur dioxide, or with another source of sulfur.

In step (c), the sulfur-containing fuel is combusted to produce heat and combustion products (generally CO₂ and H₂O), or gasified to produce heat and gasification products (generally CO, H₂, and CO₂). Step (c) may employ a fluidized-bed boiler, in some embodiments.

Heat may be recovered in step (e) for process use. For example, heat recovered in step (e) may be recycled to step (b) by recycling a portion of the intermediate liquid stream for use as the liquid or vapor solution. That is, the intermediate liquid stream may be evaporated or stripped to generate SO₂ and water (or other components) and such stream may be recycled, along with its heat content, to step (b).

In some embodiments, the cellulose-rich solids are utilized as pulp for production of a material, pellet, or consumer product. Alternatively, or additionally, the cellulose-rich solids may be combusted to produce energy. The cellulose-rich solids may also be enzymatically hydrolyzed to produce glucose.

In some embodiments, the process comprises washing the cellulose-rich solids using an aqueous wash solution, to produce a wash filtrate; and contacting at least a portion of the exhaust gas stream with the wash filtrate to dissolve the produced sulfur dioxide into the wash filtrate. In these or other embodiments, the process comprises pressing the cellulose-rich solids to produce a dewatered cellulose-rich solids and a press filtrate; and contacting at least a portion of the exhaust gas stream with the press filtrate to dissolve the produced sulfur dioxide into the press filtrate. It is also contemplated that the medium for scrubbing SO₂ could be the wash filtrate and/or press filtrate alone, i.e. not in combination with the intermediate liquid stream.

In some variations of the invention, step (b) includes extracting hemicelluloses from the feedstock in the presence of steam or hot water, to generate hemicellulose oligomers in a liquid solution. The liquid solution is then treated with the liquid or vapor solution comprising reactant sulfur dioxide or a derivative thereof. In some embodiments, the liquid or vapor solution further comprises a solvent for lignin, such as ethanol.

The sulfur dioxide that is recycled may serve one or more chemical functions. In some embodiments, the reactant sulfur dioxide, or a derivative thereof, is effective to hydrolyze hemicellulose oligomers, contained in a liquid phase, into hemicellulose monomers. In these or other embodiments, the reactant sulfur dioxide, or a derivative thereof, is effective to hydrolyze hemicellulose oligomers, contained in a solid phase, into hemicellulose monomers.

Hemicellulosic sugars may be recovered and optionally fermented to a fermentation product. If glucose is also produced from the cellulose, the glucose may also be fermented, alone or together with the hemicellulosic sugars.

Apparatus may be configured for carrying out the disclosed processes using chemical-engineering principles known in the art as well as principles disclosed in commonly owned patents and patent applications, cited below and incorporated by reference herein.

### DETAILED DESCRIPTION OF SOME EMBODIMENTS

This description will enable one skilled in the art to make and use the invention, and it describes several embodiments, adaptations, variations, alternatives, and uses of the invention. These and other embodiments, features, and advantages of the present invention will become more apparent to those skilled in the art when taken with reference to the following detailed description of the invention in conjunction with any accompanying drawings.

As used in this specification and the appended claims, the singular forms “a,” “an,” and “the” include plural referents unless the context clearly indicates otherwise. Unless defined otherwise, all technical and scientific terms used herein have the same meaning as is commonly understood by one of ordinary skill in the art to which this invention belongs. All composition numbers and ranges based on percentages are weight percentages, unless indicated otherwise. All ranges of numbers or conditions are meant to encompass any specific value contained within the range, rounded to any suitable decimal point.

Unless otherwise indicated, all numbers expressing parameters, reaction conditions, concentrations of components, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” limits the scope of a claim to the specified elements or method steps, plus those that do not materially affect the basis and novel characteristic(s) of the claimed subject matter.

With respect to the terms “comprising,” “consisting of,” and “consisting essentially of,” where one of these three terms is used herein, the presently disclosed and claimed subject matter may include the use of either of the other two terms. Thus in some embodiments not otherwise explicitly recited, any instance of “comprising” may be replaced by “consisting of” or, alternatively, by “consisting essentially of”.

The present invention, in some variations, is premised on the realization that for biorefining processes employing sulfur dioxide, sufficient quantities or concentrations of SO₂ can be economically realized by utilizing digested pulp filtrate as a scrubbing medium for an SO₂-rich combustion exhaust. The SO₂-rich combustion exhaust may derive from a coal boiler or a fluidized bed combusting lignin byproduct from the biomass hydrolysis, for example.

Several benefits may be realized in the context of this invention. First, adequate sulfur dioxide could be captured at no purchased cost for external SO₂. For example, if coal (which contains significant sulfur) is combusted for energy needs in the process, or even for a separate process, then a liquid stream may be used to economically capture the sulfur dioxide generated by sulfur oxidation. If sulfonated lignin is generated in the process and that sulfonated lignin is combusted, then SO₂ will be present in the stack gases. If all that SO₂ is recycled, there would be no need for make-up SO₂, although in practice, some make-up SO₂ would be typical.

Another potential benefit is that significant evaporative duty can be achieved in cooling stack gases. That is, when a liquid scrubbing medium (digested pulp filtrate, wash filtrate, etc.) is used to dissolve SO₂, there will also be a transfer of heat from the stack gases to the liquid medium. When that medium is to be recycled for chemical reuse, generally to a digestion step at fairly high temperature, there is a requirement for process heat. The process material and energy balance benefits.

Another possible benefit is improvement in the environmental air emissions from power boilers, since SO₂ exhaust will be reduced. Another benefit can be elimination of Tier II reportable quantities of sulfur dioxide on site. In certain embodiments, pressurized SO₂ storage is eliminated, other than reactor (digestor) volume.

Some variations of the invention are premised on the realization that sulfur dioxide may be a preferred sulfur-containing acid catalyst, or precursor thereof, for hydrolyzing biomass or components thereof, e.g. hemicellulosic extracts. There are several potential reasons, without being limited to any particular theory or hypothesis.

First, it is believed that sulfur dioxide is a more-efficient catalyst for catalyzing hydrolysis reactions to convert hemicellulose oligomers to monomers. Sulfur dioxide at ambient conditions is a gas which will have higher mass-transfer rates within a hydrolysis reactor, leading to more uniform hydrolysis chemistry. It is thought that in order for SO₂ to function as a hydrolysis catalyst, it must proceed through a reactive intermediate that contains a proton (H⁺). After the reaction step, the proton may be returned to solution and molecular SO₂ regenerated.

In particular, SO₂ in water will normally convert to some extent to sulfurous acid, H₂SO₃ (which exists in solution as H⁺ and HSO₃⁻) whose dissociated hydrogen atom may initiate the reaction. The reaction hydrolysis starts with a proton from sulfurous acid interacting rapidly with a glycosidic oxygen linking two sugar units, forming a conjugate acid. The cleavage of the C—O bond and breakdown of the conjugate acid to the cyclic carbonium ion then takes place. After a rapid addition of a molecule of water, free sugar and a proton are liberated. That proton must return to the starting acid, H₂SO₃, or to the water phase. Stoichiometrically, another way to view these reactions is that SO₂ temporarily combines with water, which is added to the sugar polymers to hydrolyze them (necessarily consuming a water molecule). In turn, the SO₂ is again available for further chemistry, or recovery from the reactor prior to the reactor contents moving downstream. Recovery is made easier since the SO₂ molecule is very volatile.

This increased efficiency owing to the inherent properties of sulfur dioxide mean that less acid may be required. This has cost advantages itself, since sulfuric acid can be expensive. Additionally, and quite significantly, less acid usage also will translate into lower costs for a base (e.g., lime) to increase the pH following hydrolysis, for downstream operations. Furthermore, less acid and less base will also mean substantially less generation of waste salts (e.g., gypsum) that may otherwise require disposal.

Another reason that sulfur dioxide may be preferred relates not to sugar hydrolysis chemistry, but to lignin chemistry. It has been surprisingly discovered, through lab-scale experiments, that acid hydrolysis of hemicellulose with sulfur dioxide leads to dramatically less lignin deposition, compared to acid hydrolysis with sulfuric acid, for the same final sugar yield.

Without being limited by any theory, it is believed that SO₂ (or HSO₃⁻) can react directly with lignin to produce sulfonated lignin (also known as lignosulfonates). The reaction of sulfur dioxide or a bisulfite ion with lignin is thought to involve acidic cleavage of ether bonds, which connect many of the constituents of lignin. The electrophilic carbocations produced during ether cleavage react with bisulfite ions to give lignosulfonates. An important site for ether cleavage is the α-carbon (carbon atom attached to the aromatic ring) of the propyl side chain of lignin. Sulfur dioxide does not tend to catalyze condensation reactions of lignin that increase molecular weight. Mechanistically, acid-catalyzed condensation and sulfonation can involve the same carbon atom, the α-carbon of the propyl group. The implication is that SO₂ or HSO₃⁻ may directly react with this carbon atom before condensation reactions can be initiated.

Also, native (non-sulfonated) lignin is hydrophobic, while lignosulfonates are hydrophilic. Hydrophilic lignosulfonates may have less propensity to clump, agglomerate, and stick to surfaces. Even lignosulfonates that do undergo some condensation and increase of molecular weight, will still have an HSO₃ group that will contribute some solubility (hydrophilic).

Another reason that sulfur dioxide may be a preferred acid catalyst, or precursor thereof, is that SO₂ can be recovered easily from solution after hydrolysis. The majority of the SO₂ from the hydrolysate may be stripped and recycled back to the reactor. Recovery and recycling translates to less lime required compared to neutralization of comparable sulfuric acid, less solids to dispose of, and less separation equipment.

Certain exemplary embodiments of the invention will now be described. These embodiments are not intended to limit the scope of the invention as claimed. The order of steps may be varied, some steps may be omitted, and/or other steps may be added. Reference herein to first step, second step, etc. is for illustration purposes only.

In some variations, the invention provides a biorefining process comprising:

(a) providing a lignocellulosic biomass feedstock;

(b) extracting, hydrolyzing, and/or digesting the feedstock, or a component thereof, with a liquid or vapor solution comprising reactant sulfur dioxide or a derivative thereof, to generate cellulose-rich solids and an intermediate liquid stream;

(c) combusting or gasifying a sulfur-containing fuel to generate heat and an exhaust gas stream comprising produced sulfur dioxide;

(d) optionally removing at least a portion of hemicellulosic sugars and/or lignin, if present, from the intermediate liquid stream;

(e) contacting at least a portion of the exhaust gas stream with the intermediate liquid stream to dissolve the produced sulfur dioxide into the intermediate liquid stream; and

(f) recycling at least some of the intermediate liquid stream back to step (b) to reuse the produced sulfur dioxide as the reactant sulfur dioxide.

In some embodiments, the sulfur-containing fuel comprises coal. In these or other embodiments, the sulfur-containing fuel comprises an intermediate stream or product derived from a biomass source, such as biochar or lignin. The biomass source may be the same as or different than the lignocellulosic biomass feedstock.

In certain embodiments, the sulfur-containing fuel comprises sulfonated lignin. The sulfonated lignin may be provided from an external process, or it may be generated in step (b) by reaction of lignin (derived from the feedstock) with the reactant sulfur dioxide, or with another source of sulfur.

In step (c), the sulfur-containing fuel is combusted to produce heat and combustion products (generally CO₂ and H₂O), or gasified to produce heat and gasification products (generally CO, H₂, and CO₂). Step (c) may employ a fluidized-bed boiler, in some embodiments.

Heat may be recovered in step (e) for process use. For example, heat recovered in step (e) may be recycled to step (b) by recycling a portion of the intermediate liquid stream for use as the liquid or vapor solution. That is, the intermediate liquid stream may be evaporated or stripped to generate SO₂ and water (or other components) and such stream may be recycled, along with its heat content, to step (b).

In some embodiments, the cellulose-rich solids are utilized as pulp for production of a material, pellet, or consumer product. Alternatively, or additionally, the cellulose-rich solids may be combusted to produce energy. The cellulose-rich solids may also be enzymatically hydrolyzed to produce glucose.

In some embodiments, the process comprises washing the cellulose-rich solids using an aqueous wash solution, to produce a wash filtrate; and contacting at least a portion of the exhaust gas stream with the wash filtrate to dissolve the produced sulfur dioxide into the wash filtrate. In these or other embodiments, the process comprises pressing the cellulose-rich solids to produce a dewatered cellulose-rich solids and a press filtrate; and contacting at least a portion of the exhaust gas stream with the press filtrate to dissolve the produced sulfur dioxide into the press filtrate. It is also contemplated that the medium for scrubbing SO₂ could be the wash filtrate and/or press filtrate alone, i.e. not in combination with the intermediate liquid stream.

In some variations of the invention, step (b) includes extracting hemicelluloses from the feedstock in the presence of steam or hot water, to generate hemicellulose oligomers in a liquid solution. The liquid solution is then treated with the liquid or vapor solution comprising reactant sulfur dioxide or a derivative thereof. In some embodiments, the liquid or vapor solution further comprises a solvent for lignin, such as ethanol.

The sulfur dioxide that is recycled may serve one or more chemical functions. In some embodiments, the reactant sulfur dioxide, or a derivative thereof, is effective to hydrolyze hemicellulose oligomers, contained in a liquid phase, into hemicellulose monomers. In these or other embodiments, the reactant sulfur dioxide, or a derivative thereof, is effective to hydrolyze hemicellulose oligomers, contained in a solid phase, into hemicellulose monomers.

Hemicellulosic sugars may be recovered and optionally fermented to a fermentation product. If glucose is also produced from the cellulose, the glucose may also be fermented, alone or together with the hemicellulosic sugars.

The biomass feedstock may be selected from hardwoods, softwoods, forest residues, industrial wastes, pulp and paper wastes, consumer wastes, or combinations thereof. Some embodiments utilize agricultural residues, which include lignocellulosic biomass associated with food crops, annual grasses, energy crops, or other annually renewable feedstocks. Exemplary agricultural residues include, but are not limited to, corn stover, corn fiber, wheat straw, sugarcane bagasse, sugarcane straw, rice straw, oat straw, barley straw, miscanthus, energy cane straw/residue, or combinations thereof

As used herein, “lignocellulosic biomass” means any material containing cellulose and lignin. Lignocellulosic biomass may also contain hemicellulose. Mixtures of one or more types of biomass can be used. In some embodiments, the biomass feedstock comprises both a lignocellulosic component (such as one described above) in addition to a sucrose-containing component (e.g., sugarcane or energy cane) and/or a starch component (e.g., corn, wheat, rice, etc.).

Various moisture levels may be associated with the starting biomass. The biomass feedstock need not be, but may be, relatively dry. In general, the biomass is in the form of a particulate or chip, but particle size is not critical in this invention.

Reaction conditions and operation sequences may vary widely. In some embodiments, the process is a variation of the AVAP® process technology which is commonly owned with the assignee of this patent application. In some embodiments, the process is a variation of the Green Power+® process technology which is commonly owned with the assignee of this patent application.

A portion or all of the sulfur dioxide may be present as sulfurous acid in the extract liquor. In certain embodiments, sulfur dioxide is generated in situ by introducing sulfurous acid, sulfite ions, bisulfite ions, combinations thereof, or a salt of any of the foregoing. Excess sulfur dioxide, following hydrolysis, may be recovered and reused.

In some embodiments, sulfur dioxide is saturated in water (or aqueous solution, optionally with an alcohol) at a first temperature, and the hydrolysis is then carried out at a second, generally higher, temperature. In some embodiments, sulfur dioxide is sub-saturated. In some embodiments, sulfur dioxide is super-saturated.

Recovering and recycling the sulfur dioxide may utilize separations such as, but not limited to, vapor-liquid disengagement (e.g. flashing), steam stripping, extraction, or combinations or multiple stages thereof

Fermentable sugars are defined as hydrolysis products of cellulose, galactoglucomannan, glucomannan, arabinoglucuronoxylans, arabinogalactan, and glucuronoxylans into their respective short-chained oligomers and monomer products, i.e., glucose, mannose, galactose, xylose, and arabinose. The fermentable sugars may be recovered in purified form, as a sugar slurry or dry sugar solids, for example. Any known technique may be employed to recover a slurry of sugars or to dry the solution to produce dry sugar solids.

In some embodiments, the fermentable sugars are fermented to produce biochemicals or biofuels such as (but by no means limited to) ethanol, isopropanol, acetone, 1-butanol, isobutanol, lactic acid, succinic acid, or any other fermentation products. Some amount of the fermentation product may be a microorganism or enzymes, which may be recovered if desired.

Any stream generated by the disclosed processes may be partially or completed recovered, purified or further treated, analyzed (including on-line or off-line analysis), and/or marketed or sold.

Apparatus may be configured for carrying out the disclosed processes using chemical-engineering principles known in the art as well as principles disclosed in commonly owned patents and patent applications, cited above and incorporated by reference herein.

In this detailed description, reference has been made to multiple embodiments of the invention and non-limiting examples relating to how the invention can be understood and practiced. Other embodiments that do not provide all of the features and advantages set forth herein may be utilized, without departing from the spirit and scope of the present invention. This invention incorporates routine experimentation and optimization of the methods and systems described herein. Such modifications and variations are considered to be within the scope of the invention defined by the claims.

All publications, patents, and patent applications cited in this specification are herein incorporated by reference in their entirety as if each publication, patent, or patent application were specifically and individually put forth herein.

Where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially.

Therefore, to the extent there are variations of the invention, which are within the spirit of the disclosure or equivalent to the inventions found in the appended claims, it is the intent that this patent will cover those variations as well. The present invention shall only be limited by what is claimed.

What is claimed is:

1. A biorefining process comprising:
(a) providing a lignocellulosic biomass feedstock;
(b) extracting, hydrolyzing, and/or digesting said feedstock, or a component thereof, with a liquid or vapor solution comprising reactant sulfur dioxide or a derivative thereof, to generate cellulose-rich solids and an intermediate liquid stream;
(c) combusting or gasifying a sulfur-containing fuel to generate heat and an exhaust gas stream comprising produced sulfur dioxide;
(d) optionally removing at least a portion of hemicellulosic sugars and/or lignin, if present, from said intermediate liquid stream;
(e) contacting at least a portion of said exhaust gas stream with said intermediate liquid stream to dissolve said produced sulfur dioxide into said intermediate liquid stream; and
(f) recycling at least some of said intermediate liquid stream back to step (b) to reuse said produced sulfur dioxide as said reactant sulfur dioxide.

2. The process of claim 1, wherein said sulfur-containing fuel comprises coal.

3. The process of claim 1, wherein said sulfur-containing fuel comprises an intermediate stream or product derived from a biomass source.

4. The process of claim 3, wherein said biomass source is said lignocellulosic biomass feedstock.

5. The process of claim 1, wherein said sulfur-containing fuel comprises sulfonated lignin.

6. The process of claim 5, wherein said sulfonated lignin is generated in step (b) by reaction of lignin derived from said feedstock, with said reactant sulfur dioxide.

7. The process of claim 1, wherein step (c) utilizes a fluidized-bed boiler.

8. The process of claim 1, wherein heat is recovered in step (e) for process use.

9. The process of claim 8, wherein heat recovered in step (e) is recycled to step (b) by recycling a portion of said intermediate liquid stream for use as said liquid or vapor solution.

10. The process of claim 1, wherein said cellulose-rich solids are utilized as pulp for production of a material, pellet, or consumer product.

11. The process of claim 1, wherein said cellulose-rich solids are combusted to produce energy.

12. The process of claim 1, wherein said cellulose-rich solids are enzymatically hydrolyzed to produce glucose.

13. The process of claim 1, said process comprising washing said cellulose-rich solids using an aqueous wash solution, to produce a wash filtrate; and contacting at least a portion of said exhaust gas stream with said wash filtrate to dissolve said produced sulfur dioxide into said wash filtrate.

14. The process of claim 1, said process comprising pressing said cellulose-rich solids to produce a dewatered cellulose-rich solids and a press filtrate; and contacting at least a portion of said exhaust gas stream with said press filtrate to dissolve said produced sulfur dioxide into said press filtrate.

15. The process of claim 1, wherein step (b) includes extracting hemicelluloses from said feedstock in the presence of steam or hot water, to generate hemicellulose oligomers in a liquid solution, wherein said liquid solution is then treated with said liquid or vapor solution comprising reactant sulfur dioxide or a derivative thereof.

16. The process of claim 1, wherein said liquid or vapor solution further comprises a solvent for lignin.

17. The process of claim 1, wherein said reactant sulfur dioxide, or a derivative thereof, is effective to hydrolyze hemicellulose oligomers, contained in a liquid phase, into hemicellulose monomers.

18. The process of claim 1, wherein said reactant sulfur dioxide, or a derivative thereof, is effective to hydrolyze hemicellulose oligomers, contained in a solid phase, into hemicellulose monomers.

19. The process of claim 1, said process further comprising recovering hemicellulosic sugars.

20. The process of claim 19, wherein said hemicellulosic sugars are fermented to a fermentation product.
